(19) United States
(12) Patent Application Publication
(10) Pub. No.: US 2026/0174784 A1
(43) Pub. Date: Jun. 25, 2026

(54) Molecular Signatures that Predict Risk for Development of Hepatic Encephalopathy

(71) Applicants: The Regents of the University of California, Oakland, CA (US); The United States Government As Represented By The Department Of Veterans Affairs, Washington, DC (US); The University of Florida Research Foundation, Incorporated, Gainesville, FL (US)

(72) Inventors: Amir Zarrinpar, San Diego, CA (US); Pieter C. Dorrestein, Oakland, CA (US); Ana Carolina Dantas Machado, Escondido, CA (US); Stephany Flores Ramos, San Diego, CA (US); Ali Zarrinpar, Gainesville, FL (US)

(21) Appl. No.: 19/124,613

(22) PCT Filed: Oct. 25, 2023
(86) PCT No.: PCT/US2023/035898

§ 371 Date: Apr. 25, 2025

Related U.S. Application Data
(60) Provisional application No. 63/380,898, filed on Oct. 25, 2022.

(51) Int. Cl. A61K 31/575; A61K 31/198

(52) U.S. Cl. CPC A61K 31/575; A61K 31/198

(57) ABSTRACT

Methods for identifying biomarkers indicative of an increased risk for developing hepatic encephalopathy (HE) following transjugular intrahepatic portosystemic shunting (TIPS) are provided. Methods include determining whether the subject has a decreased level of metabolites following transjugular intrahepatic portosystemic shunting (TIPS), and/or determining a level of intrahepatic shunting in the subject. Methods for preventing and treating a subject having or at risk of having HE identified by the methods disclosed.

### CROSS REFERENCE TO RELATED APPLICATIONS

This application claims priority to U.S. Provisional Application No. 63/380,898 filed on Oct. 25, 2022, the entire contents of which are incorporated by reference.

### GOVERNMENT SPONSORSHIP

This invention was made with government support under Grant Nos. HL148801, MH117780, and TR001442 awarded by the National Institutes of Health. The government has certain rights in the invention.

### TECHNICAL FIELD

The present invention relates generally to identifying biomarkers indicative of an increased risk for developing hepatic encephalopathy (HE) following transjugular intrahepatic portosystemic shunting (TIPS).

### BACKGROUND

Hepatic encephalopathy (HE) is a common complication of advanced liver disease, with up to 30-40% of patients developing the neurological condition as their disease progresses¹. The phenotype of HE can vary widely from mild confusion to more serious symptoms such as cognitive impairment and coma. While the etiology of HE is complex, the most prevalent theory of its pathophysiology is the inability of dysfunctional hepatocytes to process enteric neurotoxins that accumulate within the splanchnic circulation, enter the systemic circulation, and precipitate cerebral inflammation²⁻³.

In severe liver disease with refractory ascites or variceal bleeding, transjugular intrahepatic portosystemic shunt (TIPS) placement is a therapeutic intervention that can reduce pathologies caused by portal hypertension. Due to high pressure within the portal venous system and resultant hepatofugal flow, extrahepatic collateral blood vessels known as varices form to allow for portal blood return to the vena cavae and the systemic circulation⁴⁻⁵. Porto-hepatic venous collaterals, or intrahepatic shunts, can also form, although efforts to characterize this phenomenon have been sparse⁶⁻⁸.

With TIPS, a synthetic shunt connects the portal venous system to the hepatic venous system, such that blood can bypass the cirrhotic liver and reduce portal hypertension and its sequelae. Although TIPS can significantly improve the morbidity and mortality of patients with cirrhosis and increase transplant-free survival, TIPS placement is also a known risk factor for the development of HE and, thus, preexisting HE is usually a contraindication to TIPS placement. TIPS can increase the incidence of HE by 30-55% post-procedure, with 90% of affected patients developing it within the first three months⁹⁻¹².

While the association between TIPS placement and HE exacerbation is well-documented, the mechanism is poorly understood. Ammonia level is usually associated with HE, but studies examining ammonia level and HE development are conflicting. The association is highly dependent on the chronicity of hyperammonemia and varies greatly in outcome¹³⁻¹⁶. Therefore, neurotoxic compounds beyond ammonia likely contribute to HE. Microbial products such as aromatic amino acids and bile acids (BAs) are candidate neurotoxins, but it is still unclear which microbial products or metabolites induce HE onset and how TIPS placement affects the concentration of these compounds within the systemic circulation¹⁷⁻²⁰.

A promising method to identify such metabolites is untargeted metabolomics, which has been extensively applied to study microbial community function and host-microbe interactions²¹⁻²⁴. Targeted metabolomics allows for absolute quantification of specific metabolites such as amino acids and fatty acids and is limited to a few previously identified compounds for which authentic standards are available. Untargeted metabolomics, on the other hand, allows for measurement of both known and unknown metabolites, thus enabling discovery of novel metabolites and insights into biological function²⁴⁻²⁵. A key recent advance in untargeted metabolomics is the development of feature-based molecular networking (FBMN) to expand the annotation and quantification of metabolite features by enabling feature correlation analysis coupled with matching among spectral libraries and molecular networking²⁶⁻³¹.

Even for features that cannot be fully annotated, molecular networking can be used to infer compound subclass level annotation (e.g. bilirubin, bile acid) through the network connections. The potential breadth of information that can therefore be gained from untargeted metabolomics with these recently developed bioinformatic tools can lead to the identification of novel therapeutics and biomarkers³²⁻³⁷. Moreover, by obtaining serial samples from a single subject, investigators can understand what type of chemical transformations are occurring among biosynthetically related compounds and the effects these changes may have on overall physiology³⁸.

### SUMMARY OF THE INVENTION

Disclosed herein are methods for identifying biomarkers that are indicative of an increased risk for developing hepatic encephalopathy (HE) following transjugular portosystemic shunting (TIPS).

In embodiments, a method of identifying a subject with an increased risk of developing hepatic encephalopathy (HE) is provided. The method includes determining whether the subject has a decreased level of metabolites following transjugular intrahepatic portosystemic shunting (TIPS), where a decreased level of metabolites is indicative of an increased risk of developing HE. In some embodiments, metabolites are bile acid and/or glycerophosphocholine. In some embodiments, the bile acid is bilirubin. In some embodiments, the glycerophosphocholine is glycoursodeoxycholic acid (GUDCA).

In embodiments, a method of identifying a subject with an increased risk of developing hepatic encephalopathy (HE) is provided. The method includes determining a level of intrahepatic shunting in the subject, where a low level of intrahepatic shunting is indicative of an increased risk of developing HE. In some embodiments, a low level of intrahepatic shunting is correlated with an increase in portal toxins in the subject following transjugular intrahepatic portosystemic shunting (TIPS), where the increase in portal toxins increases the subject's risk of developing HE.

In embodiments, a method of identifying changes in metabolites in cirrhotic subjects undergoing transjugular intrahepatic portosystemic shunting (TIPS) is provided. The method includes collecting a pre-TIPS peripheral vein (PIV) blood sample and a pre-TIPS hepatic vein (HV) blood sample, collecting a post-TIPS PIV blood sample and a post-TIPS HV blood sample, comparing metabolites in the pre-TIPS PIV blood sample and the pre-TIPS HV blood sample with metabolites in the post-TIPS PIV blood sample and the post-TIPS HV blood sample, and identifying changes in metabolites through the comparison of the pre-TIPS PIV blood sample and the pre-TIPS HV blood sample with the post-TIPS PIV blood sample and the post-TIPS HV blood sample. In some embodiments, the metabolites in the pre-TIPS PIV blood sample and the pre-TIPS HV blood sample are compared with metabolites in the post-TIPS PIV blood sample and the post-TIPS HV blood sample by characterizing a metabolite spectral pattern in each of the pre-TIPS PIV, pre-TIPS HV, post-TIPS TIV, and post-TIPS HV blood samples. In some embodiments, changes in metabolites indicate an increased risk of developing hepatic encephalopathy (HE) following TIPS. In other embodiments, changes in metabolites indicating an increased risk of developing HE include decreased levels of bile acids in the post-TIPS PIV blood sample and the post-TIPS HV blood sample as compared to the pre-TIPS PIV blood sample and the pre-TIPS HV blood sample.

In embodiments, a method of identifying biomarkers indicating an increased risk of developing hepatic encephalopathy (HE) is provided. The method includes identifying a level of a bile acid in a subject before and after the subject undergoes transjugular intrahepatic portosystemic shunting (TIPS), where the subject has an increased risk of developing HE when a level of the bile acid following TIPS is less than a level of the bile acid before TIPS. In some embodiments, the bile acid is bilirubin.

In embodiments, a method of preventing or treating hepatic encephalopathy in a subject following transjugular intrahepatic portosystemic shunting (TIPS) is provided. The method can include increasing a level of metabolites in the subject following TIPS, where increasing the level of metabolites reduces the subject's risk for developing HE. In some embodiments, the metabolites are bile acid and/or glycerophosphocholine. In other embodiments, the bile acid is bilirubin.

In embodiments, the invention provides methods of prevention and treatment of hepatic encephalopathy (HE) in a subject, comprising administering to a subject identified to be in need thereof, by the methods described herein, an effective amount of a treatment for HE. The method can include increasing a level of metabolites in the subject, where increasing the level of metabolites reduces the subject's risk for developing HE. In some embodiments, the metabolites are bile acid and/or glycerophosphocholine. In other embodiments, the bile acid is bilirubin.

### BRIEF DESCRIPTION OF THE DRAWINGS

FIGS. 1A-1G. The effect of TIPS placement on the hepatic and peripheral metabolomes. FIG. 1A. Blood sample collection design. PIV (purple) and HV (pink) blood samples were collected pre- and post-TIPS, and following readmission for HE treatment. Visual of the location a shunt is placed during TIPS. Robust principal component analysis (RPCA) showing metabolome dissimilarities across participants in (FIG. 1B) pre-HV and post-HV samples and (FIG. 1C) pre-PIV, post-PIV, and BDc samples. FIG. 1D. The natural log ratio of bile acids to bilirubins. FIG. 1E. Heatmap showing the 87 unique metabolites that were significantly different between at least two of the three timepoints (paired Wilcoxon, FDR corrected at q-value 0.05); 25 unique metabolites significantly different pre vs. post-TIPS and 84 significantly different post-PIV vs. BDc (FDR<0.1). FIG. 1F. Pre-vs. post-PIV levels of GUDCA, (paired Wilcoxon P=0.019). FIG. 1G. Examples of bile acids that were significantly different post-PIV vs. BDc (paired Wilcoxon, FDR corrected at q-value 0.05). (GUDCA-Glycoursodeoxycholic acid; m/z 450.3 BA (mtb 176), m/z 466.3 BA (mtb 177) and m/z 432.3 BA (mtb 181) are annotated as bile acids through suspect library matching).

FIG. 2. Flowchart of patient screening, consent, admission. TIPS=transjugular intrahepatic portosystemic shunt, HE=hepatic encephalopathy, HET=hepatic encephalopathy treatment, PIV=peripheral vein, HV=hepatic vein. HE grade: 0=none; 1=mild; 2+=severe.

FIGS. 3A-3C. Feature-based molecular networking identifies metabolite features identified in plasma samples. FIG. 3A. Molecular network from all samples. Each node represents a feature or metabolite. Lines connect neighboring nodes. Colors represent different clusters with at least two nodes and one annotated feature. Subclass of main annotated clusters are shown. FIG. 3B. Percentage of feature annotation: annotated features (n=192); features within annotated cluster (n=42); unannotated features (n=361). FIG. 3C. Representation of metabolite subclass classification based on GNPS annotation.

FIG. 4. Qurro feature ranking. The positioning of bile acids vs. bilirubins in the peripheral first principal component (PC1) for the DEICODE-calculated β-diversity was used to calculate the natural log ratio.

FIGS. 5A-5E. TIPS placement effect on metabolite patterns based on HE severity.

FIG. 5A. RPCA plot showing metabolome dissimilarities across participants post-TIPS in PIV and HV samples stratified by HE grade. FIG. 5B. Portal pressure changes pre-to post-TIPS by HE grade. FIGS. 5C-5D. Comparison of the (FIG. 5C) PIV and (FIG. 5D) HV dissimilarity distances pre-to post-TIPS for each participant based on HE grade. Left: a ranked bar plot showing the (FIG. 5C) pre-to post-PIV or (FIG. 5D) pre-to post-HV dissimilarity distances for each participant. Right: boxplot showing the same data but grouped by participant HE grade. Dissimilarity is defined as the robust Aitchison β-diversity calculated with DEICODE. HE grade: 0=none (green); 1=mild (blue); 2+=severe (red). FIG. 5E theoretical framework on the relationship between intrahepatic shunting, portal neurotoxins, and development of HE.

FIGS. 6A-6D. Change from baseline (pre/post) for individual metabolites. FIG. 6A. Mean change in pre/post abundance for each metabolite based on HE grade for peripheral or hepatic vein samples. Features are ordered in ascending order for HE grade “0” group. FIG. 6B. Density plot of the change pre/post in metabolite abundances of each participant based on HE grade. Kolmogorov-Smirnov test peripheral: 0 vs 2+, P=1.3e-13; 0 vs 1, P=1.0e-03; 1 vs 2+, P=2.4e-09; hepatic: 0 vs 2+, P=5.7e-08; 0 vs 1, P=0.08; 1 vs 2+, P=2.2e-16. FIGS. 6C-6D. Change pre/post TIPS for bile acids (peripheral and hepatic vein samples) (FIG. 6C) and glycerophosphocholines (peripheral samples) (FIG. 6D) between participants based on their HE grade (0, 1, or 2+). Change pre/post is defined as the log 10 (abundancepost/abundancepre) for each participant's metabolites. HE grade: 0=none (green); 1=mild (blue); 2+=severe (red).

FIGS. 7A-7C. Levels of bile acids in plasma of TIPS participants based on HE grade. FIG. 7A. Bile acid levels and significant abundance differences in the post-TIPS peripheral blood based on HE grade. HE grade: 0=none; 1=mild; 2+=severe (Kruskal-Wallis test, FDR<0.2). FIGS. 7B-7C. Longitudinal levels of bile acids in two participants readmitted with HE grade 2+ represented by red (b, participant 11_a) and orange (c, participant 12_a) compared to participants with HE grade 0 (black line, n=4; shaded areas represent SEM).

FIGS. 8A-8H. Chemical proportionality of neighboring metabolites pre-to post-TIPS placement. FIGS. 8A-8B. Schematic representation of the (FIG. 8A) quantification table from FBMN and (FIG. 8B) data that can be deduced from neighboring metabolites, including the ChemProp score, which is calculated as the log-ratio of two neighboring metabolites pre- and post-TIPS. FIG. 8C. Network representation of hepatic ChemProp scores highlighting high-scoring clusters. FIGS. 8D-8E. Specific examples of metabolite pairs within (FIG. 8D) glycerophosphocholines (GPC) and (FIG. 8E) bilirubins clusters that display high ChemProp scores. Metabolite ID and m/z are shown for metabolites, and associated delta m/z shown for metabolite pairs. FIG. 8F. ChemProp scores for pairs of neighboring metabolites that diverged the most on their scores based on HE grade. FIGS. 8G-8H. Examples of 2 metabolite pairs, a biliverdin and glycerophosphocholine, from panel f with most divergent ChemProp scores based on HE grade. HE grade: 0=none (green); 1-mild (blue); 2+=severe (red).

FIGS. 9A-9B. Chemical proportionality pre-to post-TIPS. FIG. 9A. ChemProp score and associated Delta m/z for each pair of neighboring metabolites. FIG. 9B. Network representation of bile acids with associated IDs for individual metabolites and ChemProp scores for neighboring metabolite pairs.

### DETAILED DESCRIPTION

Disclosed herein are biomarkers indicative of an increased risk for developing hepatic encephalopathy (HE) following transjugular portosystemic shunting (TIPS).

Unless defined otherwise, all technical and scientific terms used herein have the same meanings as commonly understood by one of ordinary skill in the art to which the present disclosure belongs. Any materials and methods similar or equivalent to those described herein can be used to practice the present invention. The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J. B. Lippincott Company, 1993). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a pharmaceutical composition, and/or a method that “comprises” a list of elements (e.g., components, features, or steps) is not necessarily limited to only those elements (or components or steps), but may include other elements (or components or steps) not expressly listed or inherent to the pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the terms “a” and “an” as used herein refer to “one or more” of the enumerated components unless otherwise indicated. The use of the alternative (e.g., “or”) should be understood to mean either one, both, or any combination thereof of the alternatives.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length±15%, ±10%, ±9%, ±8%, ±7%, ±6%, +5%, ±4%, ±3%, ±2%, or ±1% about a reference quantity, level, value, number, frequency, percentage, dimension, size, amount, weight or length.

All publications, patents, and patent applications mentioned in this specification are herein incorporated by reference to the same extent as if each individual publication, patent, or patent application was specifically and individually indicated to be incorporated by reference.

In an aspect, the disclosure provides a method of treating or preventing a disease or disorder in a subject in need thereof, comprising administering to the subject an effective amount of a therapy for the disease identified by the methods described herein.

In some embodiments, administering comprises administering a therapeutically effective amount to a subject.

As used herein, the term “amount” refers to “an amount effective” or “an effective amount” of a treatment to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the subject, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the subject no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

Disclosed herein are metabolomics analyses of hepatic vein and peripheral vein blood samples from subjects undergoing TIPS, which help to understand which metabolite levels change as a result of portosystemic shunt placement and whether these changes can predict worsening HE. Standard and innovative metabolic analyses were used, which increased the overall set of annotations and allowed an exploration of the chemical modifications undergone by, and changes in levels of, the compounds after portosystemic shunt placement. Early shifts in these compounds predict the development and severity of HE and suggest the presence of intrahepatic shunting in some cirrhotic livers that is predictive of future development of HE.

HE is a serious complication of liver disease, and TIPS placement can increase its risk. The etiology and treatment of HE have historically focused on ammonia homeostasis; however, ammonia levels are not universally predictive of the degree of HE and are not reliable in guiding treatment³⁹⁻⁴⁰. In light of this incongruence, additional neurotoxins have been implicated, but no clinically applicable biomarker for HE severity has been validated. Here, the latest methods in untargeted metabolomics were used with an expanded set of annotations to characterize metabolites implicated in changes post-TIPS that could lead to the development of HE. As a result, these compounds may be used as biomarkers and potential therapeutic targets for HE.

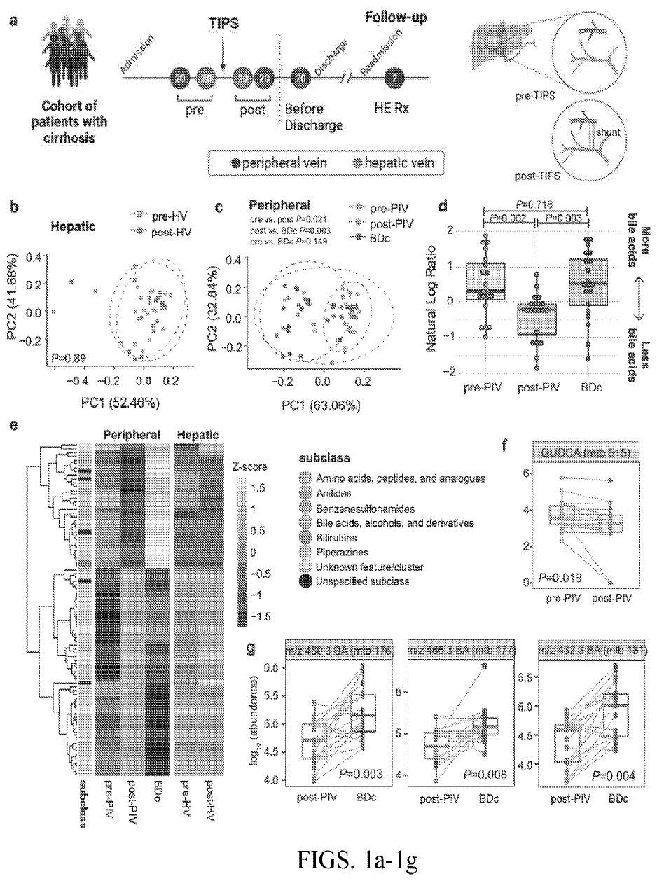

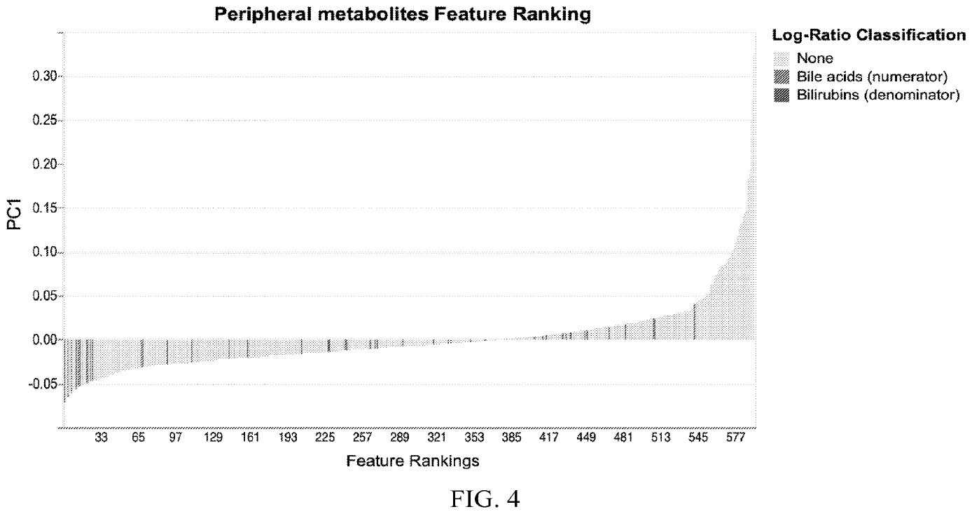

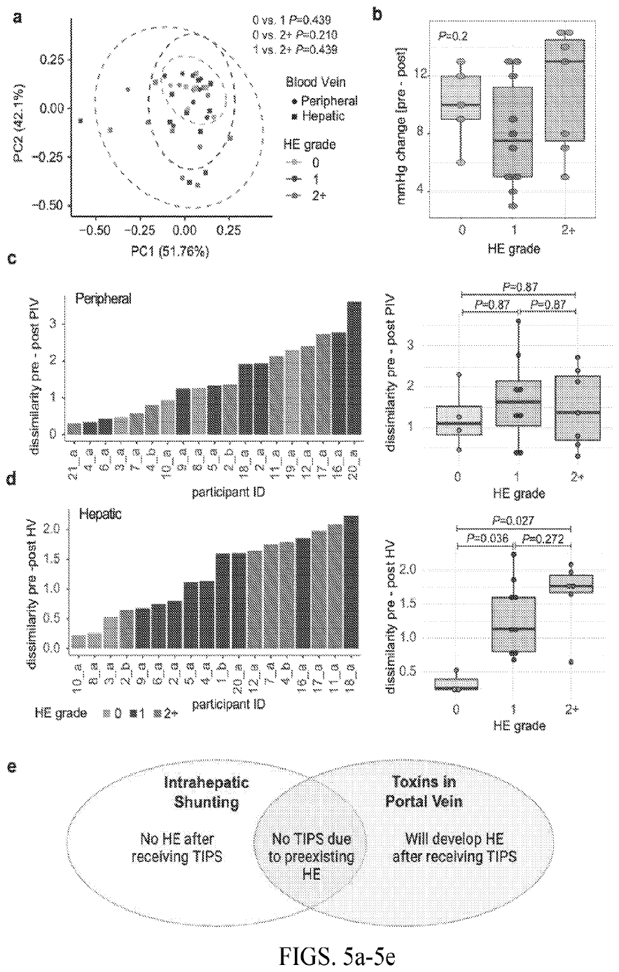

Untargeted metabolomics enable comparison of metabolites within the peripheral and hepatic circulations before and after TIPS placement. The metabolome pre-to post-TIPS changes to a greater extent in the PIV compared to the HV (FIGS. 1B-1C), and the metabolites contributing the most to these changes are bile acids (FIGS. 1D-1G, FIG. 4). Participants with the greatest degree of dissimilarity in the pre-to post-TIPS HV metabolome went on to develop HE (FIG. 5D). The higher degree of metabolite dissimilarity in HE grades 1 and 2+ implicates a functional and perhaps structural difference in participants who developed HE, namely, intrahepatic shunting. Data strongly suggests that patients with a higher degree of intrahepatic shunting either have less portal toxins or are more acclimatized to the portal toxins prior to TIPS placement, and are therefore less likely to develop HE post-TIPS. Conversely, less intrahepatic shunting pre-TIPS may lead to an overwhelming level of portal toxins in the systemic circulation post-TIPs, with subsequent development of HE as a result (FIG. 5E). These findings imply that measurement of intrahepatic shunting pre-TIPS vs. post-TIPS can assist in risk-stratifying patients susceptible to developing high-grade HE months after TIPS.

Prior methods of measuring intrahepatic shunts in patients with cirrhosis have been described but have not been updated in decades; these involve imaging using radionucleotide tagged galactose injected into the hepatic veins and pulmonary arteries, angiography similar to the access used for TIPS placement, and histologic stains for angiogenic factors after liver biopsy⁴¹⁻⁴².

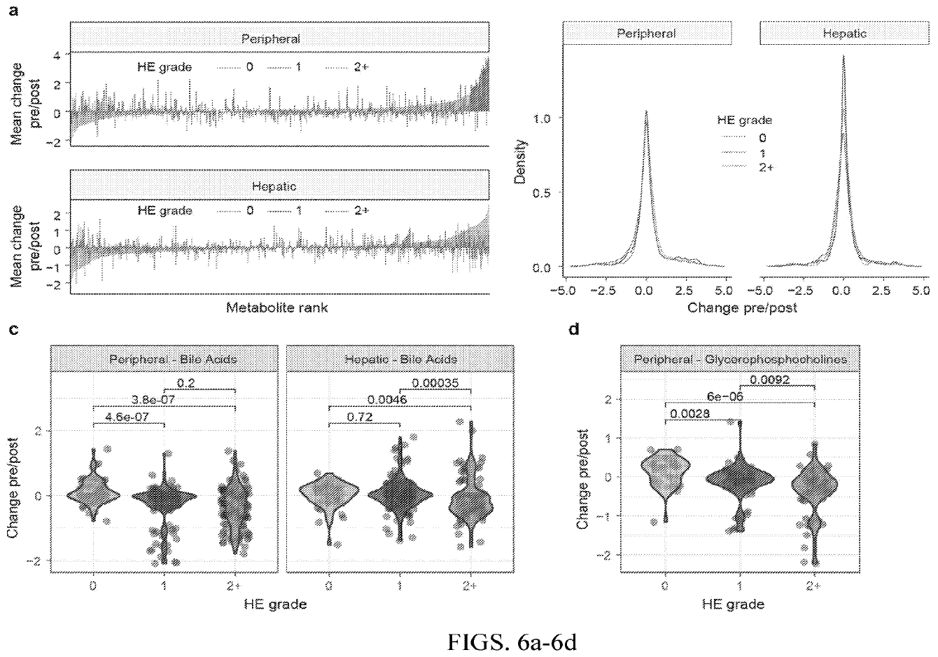

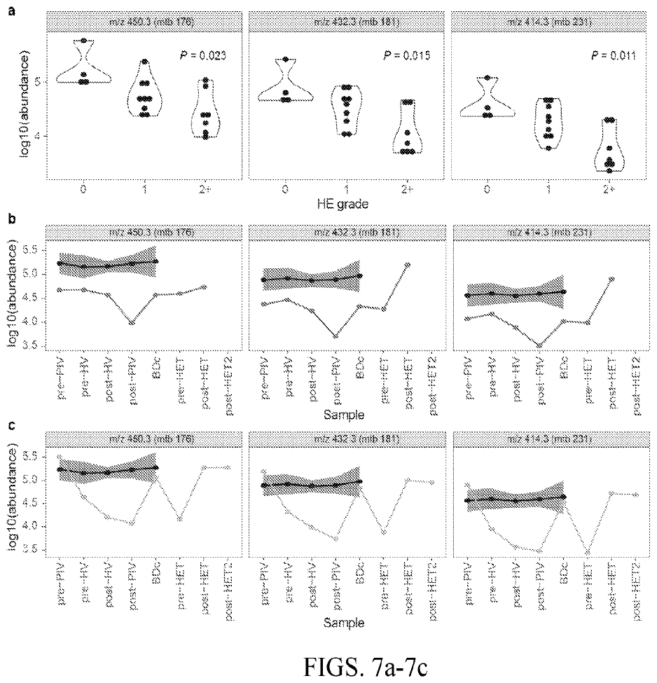

With the knowledge that intrahepatic shunting likely plays a role in post-TIPS HE and that the most significant metabolomic changes occur among bile acids pre-to post-TIPS, the relationship between bile acids and HE was explored. The change in bile acid abundances in the PIV and HV is greater in those who developed HE (FIG. 6C). This finding was confirmed upon examining bile acid changes as they relate to HE grade in the PIV, which showed three bile acids were inversely correlated with later development of HE (FIG. 7A). Patients who were hospitalized and treated for HE exacerbation had fluctuations in these specific bile acids that were consistent with their disease course (FIGS. 7B-7C). Combined, these results suggest that increased risk of developing HE post-TIPS is related to the extent the bile acid abundance pool changes immediately post-TIPS, possibly in reference to these three bile acids. The close annotation matches for these three bile acids are related to the bile acids (mtb 176), (mtb 181), and (mtb 231). Bile acids therefore play a protective role against the development of HE.

Similarly, bile acids may also have potentially protective properties in preventing or post-TIPS HE. Patients who did not develop post-TIPS HE did not demonstrate the decline in bile acids (FIG. 6C, FIG. 7), as they likely had no intrahepatic shunting at the time of TIPS and had already acclimated to the portal toxins within systemic circulation. Those with intrahepatic shunting pre-TIPS are then exposed to the alterations in glycerophosphocholine and biliverdin metabolism post-TIPS and are unable to further compensate for the portosystemic shunting. Supplementation with specific bile acids may therefore be protective against HE in patients with low degrees of intrahepatic shunting pre-TIPS.

Previous studies in animal models have shown that bile acids can give a therapeutic advantage. The neuronal oxidative damage induced by bilirubin can be inhibited by supplementation with the bile acid glycoursodeoxycholic acid (GUDCA)⁴³⁻⁴⁷. Indeed, the therapeutic, anti-inflammatory effects of GUDCA and tauroursodeoxycholic acid (TUDCA) have been described in many disorders including insulin resistance, glucose intolerance, Barrett's esophagus, retinal disease, and neurodegenerative diseases such as amyotrophic lateral sclerosis⁴⁸⁻⁵³. GUDCA directly inhibited farnesoid X receptor (FXR)⁴⁸, a ligand-mediated nuclear receptor whose activation is linked to HE and whose inhibition decreased HE symptoms in mice⁵⁴. This is the first time bile acids have been linked as being potentially protective against HE in humans.

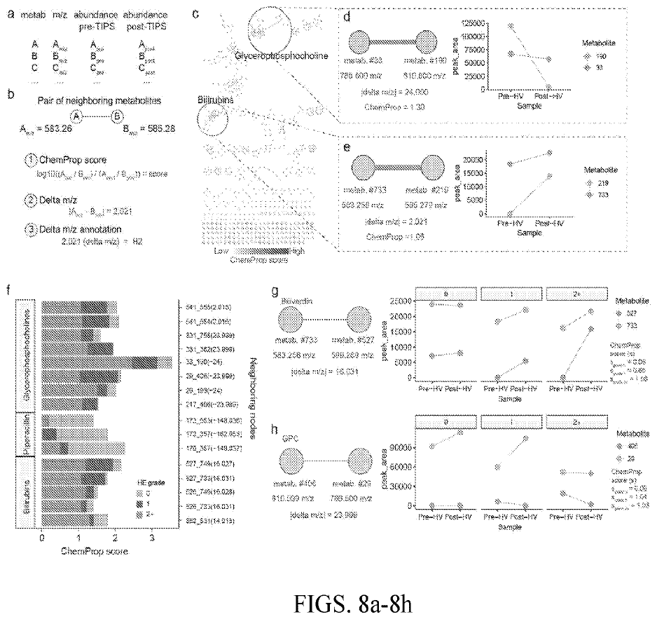

It was observed that specific bile acids decrease post-TIPS in participants with HE. With this knowledge, chemical transformations in groups of metabolites that occur pre-to post-TIPS that could be effecting these changes were assessed. FMMN was used to asses changes in neighboring metabolite nodes, while implicate potential hepatic biochemical changes. Most of these changes occur among the bilirubin and glycerophosphocholine clusters (FIGS. 8D-8G). Specifically, oxidation reactions involving biliverdin potentially occur to a greater degree in participants who developed high-grade HE. Previous studies have shown that reductions in glycerophosphocholines, a precursor to the important neurotransmitters acetylcholine and choline, are seen in patients with HIE⁵⁵⁻⁵⁷. In regards to biliverdin, there is a strong relationship between HE and free bilirubin, particularly via the albumin: bilirubin ratio, which serves as an indirect measure of plasma free bilirubin⁵⁸⁻⁶¹. These observations have led to the hypothesis that targeting free bilirubin can reduce the severity of HE in patients with liver failure.

At present, HE is managed via the enteric pharmaceuticals rifaximin and lactulose⁶²⁻⁶³. These drugs affect the composition of the gut microbiome, thus implicating microbial metabolites or products as potential agents that can influence the onset of HE, an observation supported by fecal microbiota transplant studies⁶⁴⁻⁶⁶. The relationship between the bile acid pool and gut microbiome is well-established⁶⁷⁻⁷⁰, but the role of the microbiome in HE is not thoroughly understood.

Thus, untargeted metabolomics may be used to identify changes in metabolites in cirrhotic patients undergoing TIPS. Relating these changes to HE suggested physiological differences between those that developed HE and those that did not, which suggests that intrahepatic shunting may be occurring in patients who develop HE post-TIPS. In two patients who were readmitted for HE exacerbations, decreases in bile acids were observed immediately post-TIPS, implying a protective mechanism for these metabolites. Furthermore, glycerophosphocholines and bilirubins, important neuromodulating metabolites, were shown to fluctuate within hepatic circulation post-TIPS. Ultimately, the present disclosure will assist in identifying potential biomarkers for HE, as well as targets for therapeutics in prevention and treatment of this prevalent neurologic condition.

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore, specifically point out embodiments of the present disclosure, and are not to be construed as limiting in any way the remainder of the disclosure.

### EXAMPLES

### Characterization of Metabolites Before and After TIPS Placement Implicates Plasma Bile Acid Levels

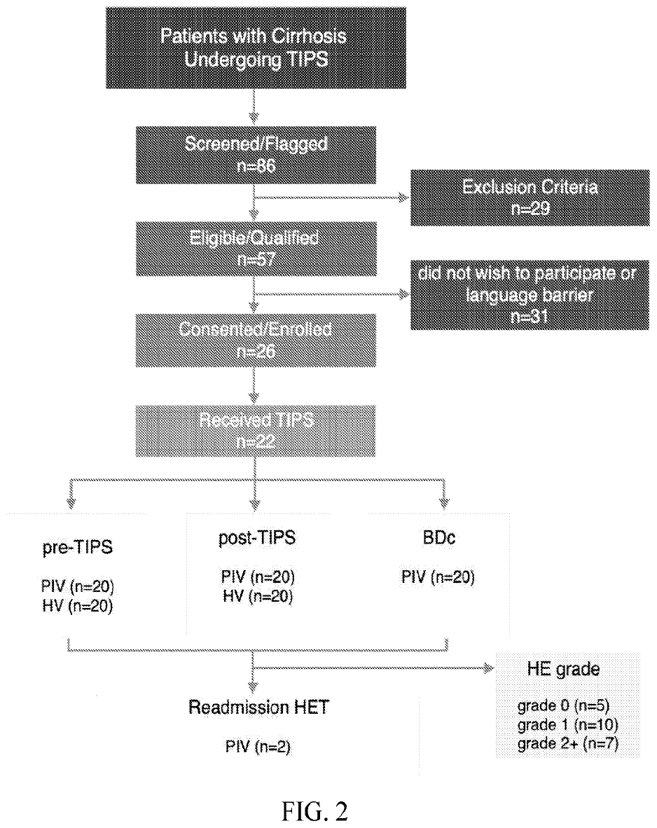

A total of 22 participants underwent TIPS placement at one of the two centers (FIG. 2). Just prior to the TIPS placement, 20 participants provided a peripheral vein (PIV) sample (FIG. 1A). During the TIPS procedure, the interventional radiologist collected hepatic vein (HV) blood for 19 participants just prior to and just after shunt placement. After the procedure but prior to admission to the post-anesthesia care unit, 19 of the participants provided another PIV blood sample. Finally, 20 of the participants provided a fasting PIV blood sample on the day of discharge with their morning labs (i.e., before discharge, BDc) (FIG. 1A). In total, 19 individuals had both pre- and post PIV, and 18 individuals had both pre- and post-PIV and HV samples, allowing for paired analysis. Participants were monitored by chart review for up to a year after their procedure and the worst HE grade during that period for each participant was noted. For two of the individuals who were readmitted with HE exacerbation during the study period, additional PIV blood samples were also obtained.

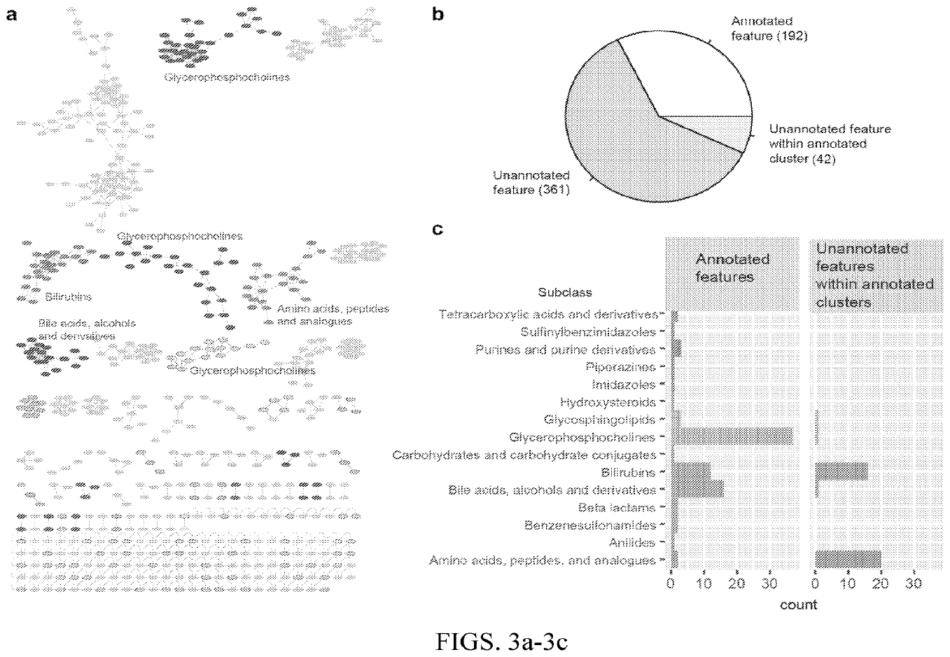

Differences in molecular distributions revealed by untargeted metabolomics associated with TIPS and HE severity were reviewed in greater detail. To do so, the metabolome of blood plasma samples collected directly from hepatic and peripheral veins pre- and post-TIPS (pre-PIV, pre-HV, post-HV, post-PIV), as well as before discharge (BDc), were characterized. Metabolite spectral patterns identified through untargeted LC/MS-MS were clustered based on similarity and annotated using feature-based molecular networking (FBMN) (FIG. 3A) through the Global Natural Products Social Molecular Networking (GNPS) platform²⁸. This resulted in the annotation of 234 features (39.3%), referred to as metabolites, among several clusters within the network (FIGS. 3B-3C). This annotation rate which greatly exceeds typical annotations (below 10%) is enabled through the use of Nearest Neighbor Suspect Spectral Library⁷².

The implications of TIPS placement on the metabolome was assessed in the peripheral and hepatic vein samples using the untargeted metabolomic data (FIGS. 1B-1D). Metabolome dissimilarities across participants (as measured using the robust Aitchison β-diversity) pre-to post-HV were not significantly different (PERMANOVA P=0.89) (FIG. 1B), but the difference reached significance between pre- and post-PIV samples (PERMANOVA P=0.021) (FIG. 1C). To account for post-TIPS changes that may take longer to manifest, the dissimilarity of BDc samples were compared to earlier PIV samples. No changes were observed pre-PIV to BDc (PERMANOVA P=0.149) but a strong change was observed post-PIV to BDc (PERMANOVA P=0.003) (FIG. 1C). These results indicate that the biggest metabolomic changes did not occur immediately after TIPS placement but rather after TIPS placement and before discharge.

Since the most significant metabolome changes occurred between the post-PIV to BDc metabolome, which metabolites are driving these shifts was assessed. To do so, Qurro was used to visualize the log-fold change (rank) of metabolites contributing to this difference⁷³. This analysis demonstrated that bile acids as a group were major contributors to the dissimilarity (FIG. 4).

To better quantify this shift in bile acids, the natural log ratio of bile acids to bilirubins was used, a group of metabolites similarly present in all three PIV timepoints (FIG. 4). This analysis demonstrated that the abundance of bile acids drops immediately after shunt placement, but abundances are restored to pre-PIV level by the time of discharge (FIG. 1D, FIG. 4).

Bile acid changes across the different time points along the TIPS procedure are also shown in paired Wilcoxon analysis of individual metabolites. Twenty-four metabolites were significantly different pre-vs. post-PIV while 12 were different pre-vs. post-HV. (FDR<0.1) (FIG. 1E). These two sets of metabolites overlap heavily; together, 25 unique metabolites are significantly different between the pre-to post-TIPS samples. The only differentially abundant metabolite with an annotation in the pre-to post-PIV analysis was glycoursodeoxycholic acid (GUDCA), which decreases in the peripheral plasma immediately following the TIPS procedure (FIG. 1F). Meanwhile, 84 metabolites were differentially abundant post-PIV to BDc, with the majority mapping to annotated bile acids (FIG. 1G). Overall, these results demonstrate that TIPS placement is associated with changes in the abundance of bile acids in peripheral circulation.

Post-TIPS HE Severity is Higher in Participants with Less Intrahepatic Shunting

The surprisingly small difference observed between the pre- and post-HV metabolome suggested considerable intrahepatic shunting is already occurring in most participants prior to undergoing TIPS. The shunting is necessarily intrahepatic because the blood being sampled comes directly from the HV and thus would not include other portosystemic shunts such as esophageal varices or splenorenal shunts.

To explore how this could affect the future development of HE, metabolome changes were assessed based on the worst HE grade within a year after the TIPS procedure. Metabolome dissimilarities across participants post-TIPS show no significant differences for any of the pairwise HE grade comparisons (PERMANOVA all pairwise P>0.05) (FIG. 5A), although there seems to be an increase in the spread of points with increasing severity. To ensure that this relationship between HE grade and variability was not due to reduction in portal hypertension after TIPS, HE grade and changes in portal pressure were compared, and a significant association was not observed (FIG. 5B).

It was also hypothesized that participants who develop more severe HE have less intrahepatic shunting at the time of their TIPS. To test this hypothesis, the shift in metabolome was compared within each participant before and after the TIPS procedure. In this analysis, those who already have significant intrahepatic shunting should have a low dissimilarity between their pre-and post-TIPS HV plasma metabolome, while those who have less shunting should have a higher dissimilarity. Within-participant dissimilarities between pre- and post-PIV metabolome did not show any difference based on HE grade (FIG. 5C). However, within-participant dissimilarities between pre- and post-HV metabolome are significantly different between participants with an HE grade of 0 versus participants with a grade of 1 or 2+ (Wilcoxon P=0.027 and P=0.036, respectively) (FIG. 5D). This change in the HV metabolome based on HE grade suggests that individuals with more dissimilarity in their metabolome after shunt placement, likely from a lack of pre-TIPS intrahepatic shunting, are at a higher risk of developing HE after the procedure (FIG. 5E).

More Severe HE Outcomes are Associated with Decreased Post-Shunt Levels of Bile Acids and Glycerosphosphocholine

Our analyses indicate that participants who developed HE had increased dissimilarity in their HV metabolome between their pre- and post-TIPS samples (FIG. 5D). To determine which metabolites are related to later development of HE, how individual metabolites changed from baseline pre-TIPS levels was analyzed (also called change from baseline and defined as log 10 (abundancepost/abundancepre) for PIV or HV samples. This analysis revealed that the change in individual metabolite levels in participants who develop post-TIPS HE (grade 1 or 2+) is quite different from that of participants with no symptoms of HE (grade 0) (FIG. 6A-6B). Overall, participants with more severe HE (2+ grade) exhibit a different distribution of the change from baseline levels of their metabolites (FIG. 6B). This is observed for both hepatic and peripheral samples, and characterized by an increased number of metabolites exhibiting lower change from baseline for participants who develop severe HE.

Out of 64 clusters (groups of related metabolites) (FIG. 2), two clusters with annotated compounds had significant differences based on HE severity: bile acids and glycerophosphocholines (FIG. 3C-3D). Larger decreases from baseline in bile acids were observed for participants with more severe HE in peripheral and hepatic vein samples, while a similar trend for glycerophosphocholine was observed in peripheral plasma (FIG. 6C-6D). Changes from baseline levels of additional clusters are significantly different between HE grades but were not associated with HE severity.

Low Levels of Post-Shunt Bile Acids are Associated with Increased Hepatic Encephalopathy Severity

It has been demonstrated that bile acids exhibit differences from baseline levels that are related to development of HE. This suggests that these metabolites play a role in HE pathophysiology (FIG. 6), and their detection in PIV may help determine patients who may be at a high risk for post-TIPS HE. A closer examination of the bile acid metabolome showed that the abundances of three bile acids in the post-PIV are significantly correlated with HE grade. All three decrease with higher HE grade, suggesting that circulating levels of these bile acids post-shunt placement are inversely associated with HE severity (FIG. 7A).

Longitudinal bile acid levels were also analyzed for two participants (participants 11_a and 12_a) who were readmitted to the hospital within one year for HE treatment (HET) following a TIPS procedure. Whether these three particular bile acid levels were also impacted at the time of readmission and treatment for HE was assessed (FIGS. 7B-7C). While these results are representative of only two participants, it is noteworthy that there is a drop in the PIV levels of these bile acids immediately after shunt placement in agreement with the results based on their later HE grade (FIGS. 7A-7C). Interestingly, upon readmission due to HE symptoms, bile acid levels are lower for participant 11_a, and HE treatment increases bile acid levels compared to pre-HET (FIG. 7B). An increase in these bile acids post-HET following readmission is also demonstrated for participant 12_a (FIG. 7C). The clinical course, demographics, and etiology for each readmitted participant revealed no overt predisposing clinical factors to explain the difference in the participants' bile acid levels. Thus, these bile acids, which are yet to be fully characterized, may play a role in HE pathophysiology or predict which patients will develop worse HE.

### Abundance and Mass Shifts in Glycerophosphocholines and Bilirubins Pre-to Post-TIPS Suggest Potential Hepatic Biotransformations That Prevent Hepatic Encephalopathy

The previous analyses demonstrated that TIPS placement disrupts the levels of specific metabolites in participants undergoing TIPS procedure (FIGS. 1C, 1E-1F), and that these changes are related to HE grade (FIGS. 6C-6D, FIG. 7A). How shunt placement affects potential chemical transformations of related compounds and how they change over time was also analyzed. To this end, an approach called chemical proportionality (ChemProp) 38 was used, which takes advantage of longitudinal abundance data associated with FBMN (FIG. 8A). The ChemProp score allows prioritization of the main changes in individual metabolite pairs based on high-scoring metabolite pairs, which can point towards important functions that are being impacted with TIPS placement (FIG. 8B) (ChemProp score). At the same time, by inspecting mass differences (FIG. 8B) (delta m/z) between related metabolites, potential biotransformations that could be involved with these shifts in abundances were highlighted (FIG. 8B) (delta m/z annotation).

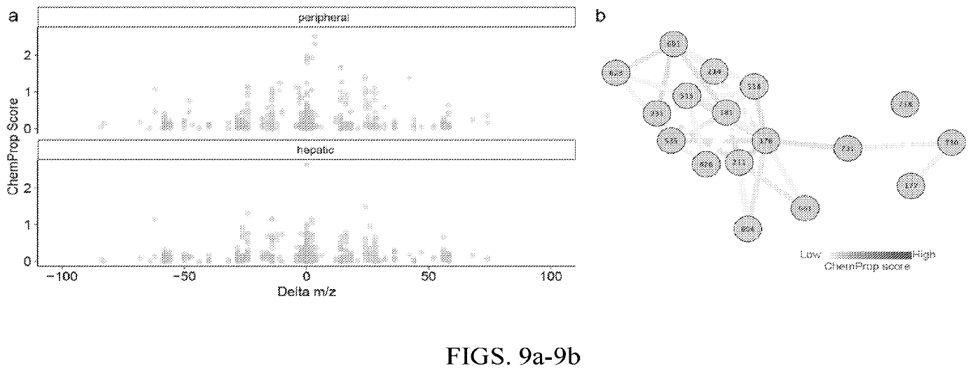

Upon calculating a ChemProp score for neighboring metabolites from hepatic or peripheral data, some of the main changes pre-to post-shunt placement were further inspected based on high-scoring pairs (FIGS. 8C, FIG. 9). The principal changes identified through ChemProp in the hepatic vein blood are related to metabolite pairs within glycerophosphocholine and bilirubin clusters, indicating the ratios between these metabolites fluctuate to a greater degree pre-to post-TIPS (FIG. 8C). For example, two diacylglycerophosphocholines with a mass change of 24.00 (C2) fluctuate to a high degree pre-to post-TIPS based on its ChemProp score (FIG. 8D). A high degree of fluctuation was also observed between two compounds within the bilirubin class was also observed, where one of the compounds matches biliverdin (FIG. 8E). The levels of these two compounds associated with a high ChemProp score and delta m/z of 2.021 suggests that biliverdin reduction or conversion of a ketone to a hydroxyl is greatly impacted pre-to post-TIPS.

When taking HE grade into account, specific sets of metabolites fluctuating at different degrees pre-to post-TIPS in patients developing HE were also identified (FIG. 8F). For example, biliverdin oxidation could be implicated as contributing to post-TIPS HE, as the fluctuation between a metabolite matching biliverdin and another metabolite with a potential delta m/z 16.031 is high for patients with HE grade 2+ (FIG. 8G). Additionally, a mass shift of 23.99 (C2) between two diacylglycerophosphocholines is high in patients with severe HE (FIG. 8H). Interestingly, most bile acid ChemProp scores are low, suggesting that the TIPS procedure itself is not altering the ratios of bile acids pre-to post-TIPS (FIG. 9).

### Cohort and Study Design

Patients scheduled to undergo elective transjugular intrahepatic portosystemic shunt (TIPS) due to severe side effects related to portal hypertension were identified by participating interventional radiology physicians prior to the procedure. Patients undergoing TIPS were screened between August 2018 and February 2019. Inclusion criteria were met if patients were cirrhotic and undergoing elective TIPS, absence of HE at the time of enrollment, age >18, not pregnant, and willing and able to consent to the study. Patients were excluded if found to have non-cirrhotic portal hypertension, other potential causes of cognitive deficits, a previous liver transplant, or prescribed medications that could cause changes in the bile acid pool (e.g. ursodiol, sequestrants). Once study participants were selected for enrollment, informed consent was obtained by a study coordinator. On the day of the procedure, peripheral vein blood (PIV) was drawn from the patients pre-TIPS. During the procedure, the interventional radiologist collected hepatic vein (HV) blood just prior to and immediately after shunt placement, described as pre-TIPS and post-TIPS HV, respectively. Hepatic pressure in mmHg was recorded during TIPS as follows: gradient pre-TIPS was measured as the difference between the hepatic wedge pressure and the right atrium; gradient post-TIPS was measured as the difference between the portal vein pressure and the right atrium pressure. All participants received one 3.375 g intravenous piperacillin-tazobactam dose intraoperatively. After the procedure but prior to admission to the post-anesthesia unit, the participants provided another peripheral vein blood sample (post-PIV). Finally, participants provided a fasting blood sample on the day of discharge with their morning labs (before discharge, BDc). Overall, the time difference between pre-PIV to post-PIV was 1-3 hours and post-PIV to BDc was 14-19 hours. The blood samples were centrifuged at 1,000 g for 15 minutes at room temperature to obtain plasma and buffy coat, which were then aliquoted and stored at −80° C. Upon discharge, subjects were monitored for up to one year for readmission related to HE, and the worst HE grade (as defined by the West Haven criteria⁷⁴ during that period for each participant was noted. Additional peripheral blood samples were collected from participants readmitted during this time period.

After the data were collected, the hospital courses of the two participants who were readmitted during the study period were examined. Participant 11_a (FIG. 6C) was a 68-year-old woman with a history of cryptogenic cirrhosis with banded esophageal varices but with no prior history of HE. She underwent TIPS procedure due to progressively worsening ascites refractory to diuretic therapy and need for intermittent paracentesis. She was readmitted to the medical intensive care unit 9 days post-TIPS with obtundation requiring intubation for airway protection. Blood was drawn on the day of admission before initiation of hepatic encephalopathy treatment (pre-HET) (FIG. 6C). Ammonia level on admission was 140 μmol/L. She was given lactulose and rifaximin for HE treatment, to which she responded well. Blood was collected for analysis on the day after treatment initiation (post-HET) (FIG. 6C). She was discharged 9 days later. However, she was readmitted within 72 hours for altered mental status. Ammonia level at the time was 169 μmol/L, but she was less encephalopathic than prior admission and did not require intubation. Her encephalopathy resolved after continuous polyethylene glycol infusion via nasojejunal tube, but was again admitted 352 days post-TIPS for HE in the setting of urosepsis.

Participant 12_a (FIG. 6B) was a 62-year-old man with a history of alcoholic cirrhosis and esophageal varices, and with no history of HE. He underwent TIPS placement with simultaneous coil embolization of a left gastric varix. On post-TIPS day 30, he became acutely altered and was admitted to a nearby hospital, where ammonia level was 123 μmol/L. He was started on lactulose, but due to worsening encephalopathy, was transferred to the tertiary facility participating in the study. On admission to the participating facility, his ammonia level was 9 μmol/L, despite ongoing encephalopathy. Blood was collected prior to hepatic encephalopathy treatment (pre-HET) (FIG. 6B). He underwent nasojejunal tube placement and was given polyethylene glycol continuously in addition to lactulose and rifaximin until his altered mentation resolved. Blood was again collected for analysis (post-HET) (FIG. 6B).

### Untargeted Metabolomics by LC-MS/MS

For untargeted LC-MS/MS, 400 μl of pre-chilled extraction solvent (100% MeOH with 1.25 μM sulfamethazine) was added to 100 μl of plasma. Samples were briefly vortexed and then incubated at −20° C. for 20 min for methanol extraction, after which they were centrifuged for 15 min at 20,000×g. Supernatant was transferred to a pre-chilled 96-well deep well plate. Samples were dried using a centrifugal low pressure system for 8h. Dried extracts were stored in sealed plates at −80° C. until analysis. Samples were resuspended in methanol (MeOH) with 1.5 μM of sulfadimethoxine, vortexed, sonicated, transferred to a shallow 96-well plate, and diluted 2λ. Untargeted metabolomics analysis was conducted using an ultrahigh-performance liquid chromatography system (UltiMate 3000; Thermo Fisher Scientific, Waltham, MA) coupled to a Maxis quadrupole time of flight (Q-TOF) mass spectrometer (Bruker Daltonics, Bremen, Germany) with a Kinetex C18 column (Phenomenex, Torrance, CA, USA). Data were collected as described in Gauglitz et al. 2020 in positive electrospray ionization mode⁷⁵. All solvents used were liquid chromatography-mass spectrometry [LC-MS]-grade (Fisher Chemical). Raw data were exported to open format.mzXML files using DataAnalysis (Bruker) and files were uploaded to the GNPS platform.

### Metabolomics Data Processing and Analysis

Feature based molecular networking (FBMN) was performed on the GNPS platform using pre-processed MZMINE2 files from LC-MS/MS experiments. In brief, MS/MS fragment ions+/−17 Da of the precursor mz were removed and the top 6 fragment ions within a +/−50 Da window were selected. Edges of the molecular network were created based on a filter of 0.65 cosine score minimum and more than 5 matched peaks. GNPS spectral libraries were used to match spectra in the network. Cytoscape was used to visualize networks. In addition, a chemical proportionality (ChemProp) approach was applied to the longitudinal data to calculate changes in abundance between neighboring nodes in order to identify changes in the abundances between every two neighboring nodes. The ChemProp score is calculated through the log 10 transformed ratio of peak area proportions of two neighboring molecular network nodes between two sequential times points³⁸. This score reflects the level of the change in abundance pre-post TIPS for a given pair of neighboring metabolites, where a high score indicates greater changes, and the sign indicates directionality. Furthermore, inspecting the mass change associated with each pair and its ChemProp strength can indicate potential biological or chemical transformations within the network, which can be visualized on a dataset scale to prioritize modification patterns⁷⁶.

### Metabolomics Statistical Analysis

To calculate metabolomic dissimilarities across participants, the robust Aitchison β-diversity was calculated using the DEICODE plugin in Qiime2 (--p-min-feature-count 10 and -p-min-sample-count 500) and plotted against robust principal component (RPCA) plots⁷⁷⁻⁷⁸. A PERMANOVA test was used to find if there were significant differences between the metabolomes of subjects based on shunt placement. To find what metabolites were driving the dissimilarity between post-PiV and BDc in the RPCA, the tool Qurro was used to find what metabolite groups were most contributing to the directionality in the first principal component (PC1)⁷⁹. Once bile acids were identified as being clustered to one side of PC1, Qurro was used to calculate the log ratio of bilirubins (a metabolite spread across PC1) to bile acids per sample. For each metabolite, a paired Wilcoxon test was run on the log 10 (abundance) at Pre-PIV vs.Post-PIV, Post-PIV vs. BDc, and Pre-PIV vs. BDc, or Pre-HV vs. Post-HV using a FDR correction of 0.1. To determine statistically significant differences in the levels of bile acids based on the worst HE grade post-TIPS (0, 1, or 2+), a Kruskal-Wallis test was run on the log 10 (abundance) of Post-PIV metabolites. To create the HE grade mixed effect models, the R package Ime4 was used to model HE grade against log 10 (abundance) PIV accounting for sex and individual person effects (mtb_abun˜HE_grade+sex+ (1|participant_id))⁸⁰. Change from baseline or change pre/post was calculated as the log 10 (abundancepost/abundancepre) for each participant's metabolite. Statistical significance was determined for: a) all changes pre/post based on HE grade using a Kolmogorov-Smirnov test (0 vs 2+; 0 vs 1; 2+vs 1); or b) changes pre/post for metabolites within each cluster using a Kruskal-Wallis test (0.2 FDR cutoff) followed by Wilcoxon test. Custom scripts were used to plot within-participant metabolome dissimilarities pre to post shunt stratified by HE grade (code repository page will be made available for publication). All analyses were performed in R version 4.1.0.

### REFERENCES

1. Madsen et al. Fecal microbiota transplantation in hepatic encephalopathy: a systematic review. Scand J Gastroenterol. 2021 May;56 (5): 560-569.
2. Wijdicks. Hepatic Encephalopathy. N Engl J Med. 2016 Oct. 27;375 (17): 1660-1670.
3. Häussinger et al. Hepatic encephalopathy. Nat Rev Dis Primers. 2022 Jun. 23;8 (1): 43.
4. Sanyal et al. Portal hypertension and its complications. Gastroenterology. 2008 May; 134 (6): 1715-28.
5. Meseeha et al. Esophageal Varices. 2023 Aug. 7. In: StatPearls [Internet]. Treasure Island (FL): StatPearls Publishing; 2023 January
6. Nakamura et al. Physiologic measurement of intrahepatic shunted blood flow by means of D-galactose-1-C₁₄. Angiology. 1971 June;22 (6): 344-53.
7. Kukla. Angiogenesis: a phenomenon which aggravates chronic liver disease progression. Hepatol Int. 2013 March;7 (1): 4-12.
8. Tanoue et al. Symptomatic intrahepatic portosystemic venous shunt: embolization with an alternative approach. AJR Am J Roentgenol. 2003 July; 181 (1): 71-8.
9. Cai et al. Psoas Muscle Density Predicts Occurrences of Hepatic Encephalopathy in Patients Receiving Transjugular Intrahepatic Portosystemic Shunts within 1 year. Cardiovasc Intervent Radiol. 2022 January;45 (1): 93-101.
10. Copelan et al. Transjugular intrahepatic portosystemic shunt: indications, contraindications, and patient work-up. Semin Intervent Radiol. 2014 September;31 (3): 235-42.
11. Boike et al. Advancing Liver Therapeutic Approaches (ALTA) Consortium. North American Practice-Based Recommendations for Transjugular Intrahepatic Portosystemic Shunts in Portal Hypertension. Clin Gastroenterol Hepatol. 2022 August;20 (8): 1636-1662.
12. Runyon. AASLD. Introduction to the revised American Association for the Study of Liver Diseases Practice Guideline management of adult patients with ascites due to cirrhosis 2012. Hepatology. 2013 April;57 (4): 1651-3.
13. Butterworth. Ammonia Removal by Metabolic Scavengers for the Prevention and Treatment of Hepatic Encephalopathy in Cirrhosis. Drugs R D. 2021 June;21 (2): 123-132.
14. Shalimar et al. Persistent or incident hyperammonemia is associated with poor outcomes in acute decompensation and acute-on-chronic liver failure. JGH Open. 2020 Feb. 28;4 (5): 843-850.
15. Ong et al. Correlation between ammonia levels and the severity of hepatic encephalopathy. Am J Med. 2003 Feb. 15;114 (3): 188-93.
16. Shawcross et al. Infection and systemic inflammation, not ammonia, are associated with Grade 3/4 hepatic encephalopathy, but not mortality in cirrhosis. J Hepatol. 2011 April;54 (4): 640-9.
17. Weiss et al. Cerebrospinal fluid metabolomics highlights dysregulation of energy metabolism in overt hepatic encephalopathy. J Hepatol. 2016 December;65 (6): 1120-1130.
18. Williams et al. A critical review of bile acids and their receptors in hepatic encephalopathy. Anal Biochem. 2022 Apr. 15; 643:114436.
19. Quinn et al. Bile acids permeabilize the blood brain barrier after bile duct ligation in rats via Rac1-dependent mechanisms. Dig Liver Dis. 2014 June;46 (6): 527-34.
20. Häussinger et al. Hepatic encephalopathy. Nat Rev Dis Primers. 2022 Jun. 23;8 (1): 43.
21. Zampieri et al. High-throughput metabolomic analysis predicts mode of action of uncharacterized antimicrobial compounds. Sci Transl Med. 2018 Feb. 21;10 (429):caal3973.
22. Johnson et al. Metabolomics: beyond biomarkers and towards mechanisms. Nat Rev Mol Cell Biol. 2016 July; 17 (7): 451-9.
23 Behnsen et al. Siderophore-mediated zinc acquisition enhances enterobacterial colonization of the inflamed gut. Nat Commun. 2021 Dec. 1;12 (1): 7016.
24. Quinn et al. Global chemical effects of the microbiome include new bile-acid conjugations. Nature. 2020 March;579 (7797): 123-129.
25. Lu et al. Metabolite Measurement: Pitfalls to Avoid and Practices to Follow. Annu Rev Biochem. 2017 Jun. 20; 86:277-304.
26. Nothias et al. Feature-based molecular networking in the GNPS analysis environment. Nat Methods. 2020 September; 17 (9): 905-908.
27 Schmid et al. Ion identity molecular networking for mass spectrometry-based metabolomics in the GNPS environment. Nat Commun. 2021 Jun. 22;12 (1): 3832.
28. Wang et al. Sharing and community curation of mass spectrometry data with Global Natural Products Social Molecular Networking. Nat Biotechnol. 2016 Aug. 9;34 (8): 828-837.
29 Xia et al. MetaboAnalyst: a web server for metabolomic data analysis and interpretation. Nucleic Acids Res. 2009 July;37 (Web Server issue): W652-60.
30. Böcker et al. SIRIUS: decomposing isotope patterns for metabolite identification. Bioinformatics. 2009 Jan. 15;25 (2): 218-24.
31. Jarmusch et al. 2020, PMID: 3280795Jarmusch et al. ReDU: a framework to find and reanalyze public mass spectrometry data. Nat Methods. 2020 September;17 (9): 901-904.
32. Wishart. Emerging applications of metabolomics in drug discovery and precision medicine. Nat Rev Drug Discov. 2016 July; 15 (7): 473-84.
33. Johnson et al. Metabolomics: beyond biomarkers and towards mechanisms. Nat Rev Mol Cell Biol. 2016 July; 17 (7): 451-9.
34. Dang et al. Untargeted Metabolomics in the Discovery of Novel Biomarkers and Therapeutic Targets for Atherosclerotic Cardiovascular Diseases. Cardiovasc Hematol Disord Drug Targets. 2018; 18 (3): 166-175.
35. Pang et al. Emerging Applications of Metabolomics in Clinical Pharmacology. Clin Pharmacol Ther. 2019 September; 106 (3): 544-556.
36. Quinn et al. Molecular Networking As a Drug Discovery, Drug Metabolism, and Precision Medicine Strategy. Trends Pharmacol Sci. 2017 February;38 (2): 143-154.
37. Bauermeister et al. Mass spectrometry-based metabolomics in microbiome investigations. Nat Rev Microbiol. 2022 March;20 (3): 143-160.
38. Petras et al. Proportionality within Molecular Networks. Anal Chem. 2021 Sep. 28;93 (38): 12833-12839.
39. Haj et al. Ammonia Levels Do Not Guide Clinical Management of Patients With Hepatic Encephalopathy Caused by Cirrhosis. Am J Gastroenterol. 2020 May; 115 (5): 723-728.
40. Lockwood. Blood ammonia levels and hepatic encephalopathy. Metab Brain Dis. 2004 December; 19 (3-4): 345-9.
41. Nakamura et al. Measurement of intrahepatic shunted blood flow by ethanol-1-14C method as compared with D-galactose-1-14C method. Tohoku J Exp Med. 1976 August; 119 (4): 345-52.
42. Tanoue et al. Symptomatic intrahepatic portosystemic venous shunt: embolization with an alternative approach. AJR Am J Roentgenol. 2003 July; 181 (1): 71-8.
43. Palmela et al. Hydrophilic bile acids protect human blood-brain barrier endothelial cells from disruption by unconjugated bilirubin: an in vitro study. Front Neurosci. 2015 Mar. 13; 9:80.
44. Silva et al. Neuritic growth impairment and cell death by unconjugated bilirubin is mediated by NO and glutamate, modulated by microglia, and prevented by glycoursodeoxycholic acid and interleukin-10. Neuropharmacology. 2012 June;62 (7): 2398-408.
45. Vaz et al. Bilirubin selectively inhibits cytochrome c oxidase activity and induces apoptosis in immature cortical neurons: assessment of the protective effects of glycoursodeoxycholic acid. J Neurochem. 2010 January; 112 (1): 56-65.
46. Brito et al. Bilirubin injury to neurons: contribution of oxidative stress and rescue by glycoursodeoxycholic acid. Neurotoxicology. 2008 March;29 (2): 259-69.
47. Fernandes et al. Glycoursodeoxycholic acid and interleukin-10 modulate the reactivity of rat cortical astrocytes to unconjugated bilirubin. J Neuropathol Exp Neurol. 2007 September;66 (9): 789-98.
48. Sun et al. Gut microbiota and intestinal FXR mediate the clinical benefits of metformin. Nat Med. 2018 December;24 (12): 1919-1929.
49. Kars et al. Tauroursodeoxycholic Acid may improve liver and muscle but not adipose tissue insulin sensitivity in obese men and women. Diabetes. 2010 August;59 (8): 1899-905.
50. Goldman et al. Protetive effects of glycoursodeoxycholic acid in Barrett's esophagus cells. Dis Esophagus. 2010 February;23 (2): 83-93.
51. Daruich et al. Review: The bile acids urso- and tauroursodeoxycholic acid as neuroprotective therapies in retinal disease. Mol Vis. 2019 Oct. 14; 25:610-624.
52. Zangerolamo et al. The bile acid TUDCA and neurodegenerative disorders: An overview. Life Sci. 2021 May 1; 272:119252.
53. Khalaf et al. Tauroursodeoxycholic acid: a potential therapeutic tool in neurodegenerative diseases. Transl Neurodegener. 2022 Jun. 4;11 (1): 33.
54. McMillin et al. FXR-Mediated Cortical Cholesterol Accumulation Contributes to the Pathogenesis of Type A Hepatic Encephalopathy. Cell Mol Gastroenterol Hepatol. 2018 Mar. 6;6 (1): 47-63.
55. Bluml et al. Proton-decoupled 31P magnetic resonance spectroscopy reveals osmotic and metabolic disturbances in human hepatic encephalopathy. J Neurochem. 1998 October; 71 (4): 1564-76
56. Asaki et al. Neurochemistry of hyponatremic encephalopathy evaluated by MR spectroscopy. Brain Dev. 2020 November;42 (10): 767-770.
57. Córdoba et al. 1H magnetic resonance in the study of hepatic encephalopathy in humans. Metab Brain Dis. 2002 December; 17 (4): 415-29.
58. Li et al. Pathological Significance and Prognostic Roles of Indirect Bilirubin/Albumin Ratio in Hepatic Encephalopathy. Front Med (Lausanne). 2021 Aug. 30; 8:706407.
59. Mohammed et al. Albumin-Bilirubin Score: An Accurate Predictor of Hepatic Decompensation in High-Risk Patients Undergoing Transarterial Chemoembolization for Hepatocellular Carcinoma. J Vasc Interv Radiol. 2018 November;29 (11): 1527-1534.el.
60 Lin et al. Efficacy of albumin-bilirubin score to predict hepatic encephalopathy in patients underwent transjugular intrahepatic portosystemic shunt. Eur J Gastroenterol Hepatol. 2021 Jun. 1;33 (6): 862-871.
61. Hou et al. Diagnostic value of albumin-bilirubin grade combined with serum ammonia in cirrhosis with hepatic encephalopathy. Zhonghua Yi Xue Za Zhi. 2018 Jan. 9;98 (2): 127-131.
62. Kimer et al. Systematic review with meta-analysis: the effects of rifaximin in hepatic encephalopathy. Aliment Pharmacol Ther. 2014 July;40 (2): 123-32.
63. Gluud et al. Non-absorbable disaccharides versus placebo/no intervention and lactulose versus lactitol for the prevention and treatment of hepatic encephalopathy in people with cirrhosis. Cochrane Database Syst Rev. 2016 May 6;2016 (5): CD003044.
64. Madsen et al. Fecal microbiota transplantation in hepatic encephalopathy: a systematic review. Scand J Gastroenterol. 2021 May;56 (5): 560-569.
65. Moran et al. Current approach to treatment of minimal hepatic encephalopathy in patients with liver cirrhosis. World J Gastroenterol. 2021 Jun. 14;27 (22): 3050-3063.
66. Iqbal et al. A Comprehensive Review Evaluating the Impact of Protein Source (Vegetarian vs. Meat Based) in Hepatic Encephalopathy. Nutrients. 2021 Jan. 26;13 (2): 370.
67. Schneider et al. Role of bile acids in the gut-liver axis. J Hepatol. 2018 May;68 (5): 1083-1085.
68. Tripathi et al. The gut-liver axis and the intersection with the microbiome. Nat Rev Gastroenterol Hepatol. 2018 July; 15 (7): 397-411.
69. Ramírez-Pérez et al. The Role of the Gut Microbiota in Bile Acid Metabolism. Ann Hepatol. 2017 November; 16 (Suppl. 1: s3-105.): s15-s20.
70. Ridlon et al. Bile acids and the gut microbiome. Curr Opin Gastroenterol. 2014 May;30 (3): 332-8.
71. Sumner et al. Proposed minimum reporting standards for chemical analysis Chemical Analysis Working Group (CAWG) Metabolomics Standards Initiative (MSI). Metabolomics. 2007 September;3 (3): 211-221.
72. Bittremieux et al. Open Access Repository-Scale Propagated Nearest Neighbor Suspect Spectral Library for Untargeted Metabolomics. bioRxiv. 2023.
73. Fedarko et al. Visualizing 'omic feature rankings and log-ratios using Qurro. NAR Genom Bioinform. 2020 June;2 (2):lqaa023.
74. Weissenborn. Hepatic Encephalopathy: Definition, Clinical Grading and Diagnostic Principles. Drugs. 2019 February;79 (Suppl 1): 5-9.
75. Gauglitz et al. Metabolome-Informed Microbiome Analysis Refines Metadata Classifications and Reveals Unexpected Medication Transfer in Captive Cheetahs. mSystems. 2020 Mar. 10;5 (2): e00635-19.
76. Hartmann et al. Meta-mass shift chemical profiling of metabolomes from coral reefs. Proc Natl Acad Sci USA. 2017 October 31;114 (44): 11685-11690.
77. Martino et al. A Novel Sparse Compositional Technique Reveals Microbial Perturbations. mSystems. 2019 Feb. 12;4 (1): e00016-19.
78. Bolyen et al. Reproducible, interactive, scalable and extensible microbiome data science using QIIME 2. Nat Biotechnol. 2019 August;37 (8): 852-857. doi: 10.1038/s41587-019-0209-9. Erratum in: Nat Biotechnol. 2019 September;37 (9): 1091.
79. Fedarko et al. Visualizing 'omic feature rankings and log-ratios using Qurro. NAR Genom Bioinform. 2020 June;2 (2):lqaa023.
80. Bates, et al. Fitting Linear Mixed-Effects Models Using Ime4. Journal of Statistical Software, 2015; 67 (1), 1-48.

1. A method of identifying a subject with an increased risk of developing hepatic encephalopathy (HE) comprising:
determining whether the subject has a decreased level of metabolites following transjugular intrahepatic portosystemic shunting (TIPS);
wherein a decreased level of metabolites is indicative of an increased risk of developing HE.

2. The method of claim 1, wherein the metabolites are bile acid and/or glycerophosphocholine.

3. The method of claim 2, wherein the bile acid is glycoursodeoxycholic acid (GUDCA).

4. The method of claim 1, wherein the metabolites are modified bilirubins and glycerophosphocholines.

5. A method of identifying a subject with an increased risk of developing hepatic encephalopathy (HE) comprising:
determining a level of intrahepatic shunting in the subject;
wherein a low level of intrahepatic shunting is indicative of an increased risk of developing HE.

6. The method of claim 5, wherein a low level of intrahepatic shunting is correlated with an increase in portal toxins in the subject following transjugular intrahepatic portosystemic shunting (TIPS), and wherein the increase in portal toxins increases the subject's risk of developing HE.

7. A method of identifying changes in metabolites in cirrhotic subjects undergoing transjugular intrahepatic portosystemic shunting (TIPS) comprising:
collecting a pre-TIPS peripheral vein (PIV) blood sample and a pre-TIPS hepatic vein (HV) blood sample;
collecting a post-TIPS PIV blood sample and a post-TIPS HV blood sample;
comparing metabolites in the pre-TIPS PIV blood sample and the pre-TIPS HV blood sample with metabolites in the post-TIPS PIV blood sample and the post-TIPS HV blood sample; and
identifying changes in metabolites through the comparison of the pre-TIPS PIV blood sample and the pre-TIPS HV blood sample with the post-TIPS PIV blood sample and the post-TIPS HV blood sample.

8. The method of claim 7, wherein metabolites in the pre-TIPS PIV blood sample and the pre-TIPS HV blood sample are compared with metabolites in the post-TIPS PIV blood sample and the post-TIPS HV blood sample by characterizing a metabolite spectral pattern in each of the pre-TIPS PIV, pre-TIPS HV, post-TIPS TIV, and post-TIPS HV blood samples.

9. The method of claim 7, wherein changes in metabolites indicate an increased risk of developing hepatic encephalopathy (HE) following TIPS.

10. The method of claim 8, wherein changes in metabolites indicating an increased risk of developing HE include decreased levels of bile acids in the post-TIPS PIV blood sample and the post-TIPS HV blood sample as compared to the pre-TIPS PIV blood sample and the pre-TIPS HV blood sample.

11. A method of identifying biomarkers indicating an increased risk of developing hepatic encephalopathy (HE) comprising:
identifying a level of a bile acid in a subject before and after the subject undergoes transjugular intrahepatic portosystemic shunting (TIPS);
wherein the subject has an increased risk of developing HE when a level of the bile acid following TIPS is less than a level of the bile acid before TIPS.

12. The method of claim 11, wherein the bile acid is GUDCA.

13. A method of preventing or treating hepatic encephalopathy in a subject following transjugular intrahepatic portosystemic shunting (TIPS) comprising:
increasing a level of metabolites in the subject following TIPS;
wherein increasing the level of metabolites reduces the subject's risk for developing HE.

14. The method of claim 13, wherein the metabolites are bile acid and/or glycerophosphocholine.

15. The method of claim 14, wherein the bile acid is GUDCA.

16. The method of claim 13, wherein the metabolites are modified bilirubins and glycerophosphocholines.

17. The method of claim 7, wherein the metabolites are modified bilirubins and glycerophosphocholines.
